(19) United States
(12) Patent Application Publication
(10) Pub. No.: US 2023/0392116 A1
(43) Pub. Date: Dec. 7, 2023

(54) METHOD FOR DIFFERENTIATING CORNEAL ENDOTHELIAL CELL-LIKE CELLS FROM PLURIPOTENT STEM CELLS

(71) Applicant: StemSight Oy, Lempaeaelae (FI)

(72) Inventors: Pyry Grönroos, Tampere University, Finland (FI); Heli Skottman, Tampere University, Finland (FI); Tanja Ilmarinen, Tampere University, Finland (FI)

(21) Appl. No.: 18/044,061

(22) PCT Filed: Sep. 1, 2021
(86) PCT No.: PCT/FI2021/050588

§ 371 Date: Mar. 3, 2023

(30) Foreign Application Priority Data
Sep. 3, 2020 (FI) 20205857

(51) Int. Cl. C12N 5/079

(52) U.S. Cl. CPC C12N 5/0621; C12N 2500/38; C12N 2501/15; C12N 2506/02; C12N 2501/415; C12N 2506/45; C12N 2501/727

(57) ABSTRACT

The present invention relates to differentiation of corneal endothelial cell (CEC)-like cells from pluripotent stem cells. The invention also relates to CEC-like cells obtainable by the differentiation method, to uses thereof and to preparations comprising the same.

### TECHNICAL FIELD

The present invention relates to differentiation of corneal endothelial cell (CEC)-like cells from pluripotent stem cells. The invention also relates to CEC-like cells obtainable by the differentiation method, to therapeutic use thereof and to a preparation comprising the same.

### BACKGROUND

Human corneal endothelium is a thin cell layer in the innermost side of the cornea. It keeps the eyesight clear by pumping fluids out of the cornea. Owing to its inability to regenerate itself in the human eye, damaged corneal endothelium results in the swelling of the cornea causing both blindness and pain. Currently, the only clinically relevant treatment is corneal transplantation, which requires cadaveric donors. Unfortunately, there is a massive shortage of donor corneas.

Attempts have been made to differentiate corneal endothelial cell (CEC)-like cells from pluripotent stem cells. For example, Zhang et al. (Stem Cells Dev. 2014; 23(12):1340-54) derived CEC-like cells from human embryonic stem cells (hESCs) first by co-culturing them with corneal stroma cells to obtain periocular mesenchymal precursors (POMPs). CEC-like cells were then derived from POMPs using undefined lens epithelial cell-conditioned medium. Also McCabe et al. (PLoS One. 2015; 10(12): e0145266) used hESCs to differentiate CEC-like cells by creating a two-step generation procedure using chemically more defined method by first using Dual Smad inhibition with a TGF beta signalling blocker (SB431542) and Noggin, followed by Wnt inhibition by using platelet-derived growth factor B (PDGF-BB), Dickkopf-related protein 2 (DKK-2) and basic fibroblast growth factor (bFGF) to produce CEC-like cells. Wagoner et al. Biol Open. 2018; 7(5):1-10) derived CEC-like cells from human induced pluripotent stem cells (hiPSCs) by using a slightly modified McCabe's protocol. Zhao and Afshari (Invest Ophthalmol Vis Sci. 2016; 57(15):6878-84) differentiated CEC-like cells via a three-step method containing first dual smad inhibition with SB431542 and LDN193189 and Wnt inhibitor IWP2 to produce eye field stem cells. Then they used CHIR99021 to produce ocular neural crest stem cells and at the last step, they used SB431542 and ROCK inhibitor H-1125 to differentiate CEC-like cells. In U.S. Pat. No. 9,347,042, Shimmura et al. demonstrated differentiation of CEC-like cells from hiPSC-derived neural crest stem cells in an adherent culture by using BIO (6-Bromoindirubin-3′-oxime), all-trans-retinoic acid (ATRA), TGFb2, insulin and Y-27632 as inductive agents, whereas in U.S. Pat. No. 10,501,725, Shimmura et al. demonstrated the same in a suspension culture by using N2 supplement, epidermal growth factor (EGF), bFGF, ATRA, BIO and Y-27632.

Despite existing differentiation methods, there is still a need for a fast and simple protocol for producing CEC-like cells in large quantities in a reproducible manner.

### SUMMARY

An object of the present invention is to provide a fast and simple method of producing corneal endothelial cell (CEC)-like cells from pluripotent stem cells so as to overcome problems associated with existing differentiation protocols. This object is achieved by the method which is characterized by what is stated in the independent claims. Preferred embodiments of the invention are disclosed in the dependent claims.

Accordingly, the invention provides a method of producing CEC-like cells from pluripotent stem cells, wherein the method comprises the steps of a) culturing the pluripotent stem cells in the presence of at least one transforming growth factor beta (TGF-beta) inhibitor, at least one Wingless-related integration site (Wnt) activator and retinoic acid, and b) culturing cells from step a) in the presence of at least one TGF-beta inhibitor and at least one Wnt activator, but in the absence of or decreasing concentration of retinoic acid, thereby producing CEC-like cells. In some embodiments, the TGF-beta inhibitor is selected from the group consisting of SB431542 and SB505124. In some embodiments, the Wnt activator is selected from the group consisting of glycogen synthase kinase 3 (GSK3) inhibitors and proteins of R-spondin family. In some embodiments, the GSK3 inhibitor is CHIR99021,

In addition, the invention provides CEC-like cells obtainable by the present differentiation method.

Also provided is use of TGF-beta inhibitor, Wnt activator and retinoic acid in combination for inducing CEC-like cells from pluripotent stem cells.

In a further aspect, the invention provides the present CEC-like cells for use in the treatment of corneal endothelial dysfunction or for use in drug development.

Also provided is a preparation, such as a pharmaceutical preparation, comprising the present CEC-like cells and a solution, carrier, adjuvant and/or excipient, preferably a pharmaceutically acceptable solution, carrier, adjuvant and/or excipient.

Further aspects, embodiments, objects, details and advantages of the invention are set forth in the following drawings, detailed description and examples.

### BRIEF DESCRIPTION OF THE DRAWINGS

The accompanying drawings illustrate several embodiments of the disclosed subject matter, and together with the description, serve to explain principles of the disclosed differentiation induction method.

FIG. 1 shows phase contrast light microscope images of the morphology of hPSC-CEC cells on day 12 with or without RA for the three first days of the culture. The black arrows indicate dome structures present in the cell monolayer, indicating pumping activity. Corresponding results were obtained using Regea 08/017 hESC line. Images taken with Nikon Eclipse TE2000-S using 4× and 10× magnification.

FIG. 2 shows immunofluorescence images of the expression of ZO-1, Na+/K+-ATPase and CD166 on day 12 of the hPSC-CEC differentiation protocol with or without RA for the first three days of the culture. Corresponding results were obtained using Regea 08/017 hESC line. Images taken with Olympus IX51, using 10× magnification.

FIG. 3 shows an immunofluorescence image of the expression of Na+/K+-ATPase in cultured primary human corneal endothelial cells. Corresponding results were obtained from the cornea of a 22-year old donor. Image taken with Olympus IX51, using 10× magnification.

FIG. 4 shows an immunofluorescence image of CEC-like cells produced by the present method, demonstrating lack of OCT3/4 (POU5F1) expression. Bright spots on the image are residues of secondary antibody aggregates and not associated with the cells. Corresponding results were obtained using Regea 08/017 hESC line. Image taken with Olympus IX51, using 10× magnification.

FIG. 5 illustrates results obtained from qPCR analyses, demonstrating lack of OCT3/4 (POU5F1) expression and increased PITX2, FOXC1, SLC4A4 and AQP1 expression during the 6 days of differentiation. Due to technical error AQP1 is missing in the day 0 sample.

### DETAILED DESCRIPTION

The present invention provides a method of producing corneal endothelial cell (CEC)-like cells from pluripotent stem cells. Also provided are CEC-like cells obtainable by the method, preparations comprising the CEC-like cells as well as various uses thereof.

As used in the specification and in the appended claims, the singular expressions “a”, “an” and “the” mean one or more. Thus, a singular noun, unless otherwise specified, carries also the meaning of the corresponding plural noun.

Briefly, the present invention is based on use of three differentiation induction agents, namely a TGF-b inhibitor, a Wnt activator and retinoic acid, in a schedule described below.

### Cells

As used herein, the term “pluripotent stem cell” (PSC) refers to any stem cell having the potential to differentiate into all cell types of a human or animal body, not including extra-embryonic tissues. These stem cells include both embryonic stem cells (ESCs) and induced pluripotent cells (iPSCs). Hence, cells suitable for use in the present invention include stem cells selected from iPSCs and ESCs. The term encompasses also genetically modified PSCs, such as human leukocyte antigen (HLA)-modified PSCs. Accordingly, genetically modified PSCs, including genetically modified iPSCs and ESCs, may be employed in some embodiments of the invention.

Human pluripotent stem cells (hPSCs) are preferred and they include human iPSCs (hiPSCs) and human ESCs (hESCs). ESCs, especially hESCs, are of great therapeutic interest because they are capable of indefinite proliferation in culture and are thus capable of supplying cells and tissues for replacement of failing or defective human tissue. However, producing corneal endothelial cells from human embryonic stem cells may meet ethical challenges. According to an embodiment of the present invention, human embryonic stem cells may be used with the proviso that the method itself or any related acts do not involve destruction of human embryos.

Induced pluripotent stem cells, commonly abbreviated as iPS cells or iPSCs are a type of pluripotent stem cell artificially derived from a non-pluripotent cell, typically an adult somatic cell, by inducing a forced expression of specific genes by means and methods well known in the art. An advantage of using iPS cells is that no embryonic cells have to be used at all, so ethical concerns can be avoided. A further advantage is that production of patient-specific cells without immunorejection problems is enabled by employing iPSC technology. Therefore, according to an embodiment of the present invention, use of iPS cells is preferred. For clinical use, hiPS cells are preferred.

Induced pluripotent stem cells are similar to natural pluripotent stem cells, such as embryonic stem cells, in many aspects. Exemplary aspects include the expression of certain stem cell genes and proteins, chromatin methylation patterns, doubling time, embryoid body formation, teratoma formation, viable chimera formation, and potency and differentiability, but the full extent of their relation to natural pluripotent stem cells is still being assessed. Induced pluripotent cells are typically made from adult skin cells, blood cells, stomach or liver, although other alternatives may be possible. Those skilled in the art are familiar with the potential of iPS cells for research and therapeutic purposes.

Non-limiting examples of pluripotency markers include POU class 5 homeobox 1 (POU5F1, OCT3/4), as is well known in the art.

As used herein, the term “primary corneal endothelial cells” (CECs) refers to cells of the corneal endothelium, a monolayer of cells on the inner surface of the cornea. CECs plays several essential roles in corneal homeostasis and are specialized in regulating corneal hydration and transparency.

As used herein, the term “CEC-like cells” refers to cells obtainable through differentiation from PSCs and having essential characteristics of primary CECs. Accordingly, the term “hPSC-CEC”, as used herein, refers to CEC-like cells differentiated from human pluripotent stems with the present differentiation induction methods.

Primary CECs and the present CEC-like cells are characterized by their ability to form a monolayer of uniformly sized cells having a predominantly hexagonal apical surface and an irregular basal surface. Tight junctions play a role in forming and maintaining the hexagonality, and are needed to maintain relative dehydration of the cornea. Indeed, primary CECs and the present CEC-like cells are characterized by the expression of tight junction proteins, such as zonula occludens-1 (ZO-1).

In addition, primary CECs and the present CEC-like cells are equipped with different types of enzymatic pumps which create ionic gradients between the cornea and the aqueous humor, and are responsible for permanent extraction of water from the stroma. Non-limiting examples of corneal endothelial pump markers include ATPase Na+/K+ transporting subunit alpha 1 (ATP1A1) and solute carrier family 4 member 4 (SLC4A4).

Further CEC markers include, but are not limited to, CD166, and aquaporin 1 (AQP1).

In some embodiments of the invention, differentiation of CEC-like cells from PSCs is achieved through neural crest cells (NCCs), as judged by the emergence of NCC-specific markers, although it may be difficult to pinpoint a time point at which the PSCs have differentiated into NCCs which are then further differentiated into CEC-like cells.

As used herein, the term “neural crest cells” (NCCs) refers to a transient group of cells unique to vertebrates that arise from the embryonic ectoderm germ layer, and in turn give rise to diverse cell lineages. The neural crest can be divided into four main functional domains, which include the cranial neural crest, trunk neural crest, vagal and sacral neural crest, and cardiac neural crest. During corneal development, the cranial NCCs give rise to the corneal endothelium and stroma.

NCC-specific markers are well known in the art. Non-limiting examples of markers specific for cranial NCCs include AP2α, AP2β and nerve growth factor receptor (NGFR, p75). In addition, low or absent expression of Pax6 is characteristic in cranial NCCs.

Moreover, non-limiting examples of periocular mesenchyme markers occurring in late NCCs and early CECs include paired like homeodomain 2 (PITX2) and forkhead box C1 (FOXC1).

In accordance with the above, CEC-like cells obtainable by the production method of the invention express at least one tight junction marker, at least one pump marker and at least one CEC marker. In some embodiments, the CEC-like cells express one or more markers selected from the group consisting of ZO-1, ATP1A1, SLC4A4, CD166, AQP1, PITX2 and FOXC1.

The presence of cell type-specific markers, such as those listed above, may be quantified by any available technique suitable for this purpose including, but not limited to, quantitative PCR (qPCR), immunofluorescence and flow cytometry, such as fluorescence activated cell sorting (FACS).

### Differentiation Induction Method and Induction Agents

In the present differentiation method, PSCs are first cultured in the presence of a TGF-b inhibitor, a Wnt activator and retinoic acid (RA), then in the present of the TGF-beta inhibitor and the Wnt activator only or in a decreasing concentration of RA.

In a first step of the present method, PSCs are cultured in the presence of a TGF-beta inhibitor, Wnt activator and retinoic acid, preferably for a period of time allowing emergence of CEC-like cells.

Without being limited to any theory or mechanism of action, experiments leading to the present invention indicated that SB431542 and CHIR99021 as differentiation induction agents contributed to emergence of NCC markers. It is to be understood that the exact timing of said emergence may depend on different variables, such as the TGF-beta inhibitor and Wnt activator species employed and concentrations thereof in the cell culture medium.

In some embodiments, when the culture medium contained about 10 μM SB431542 and about 4 μM CHIR99021 as the TGF-beta inhibitor and Wnt activator, respectively, NCC markers appeared around day 3 of the differentiation induction method.

Retinoic acid, in turn, contributed to the formation of CEC-like cells and mesenchyme-like areas positive with periocular mesenchyme markers (e.g. PITX2 and FOXC1) from day 5 onwards. Moreover, RA induced the formation of dome-like structures indicating barrier properties and pumping activities in the cells. These features were not present if the cells were cultured in the absence of RA (i.e. in the presence of SB431542 and CHIR99021 only).

In some embodiments, PSCs are cultured in the presence of TGF-beta inhibitor, Wnt activator and RA from about 3 days to about 10 days, preferably from about 3 days to about 5 days. In some embodiments, PSCs are cultured in the presence of SB431542, CHIR99021 and RA from about 3 days to about 10 days, preferably from about 3 days to about 5 days. Too long an exposure to RA results in the formation on vacuoles and emergence of adipocyte-like cells, both of which features are to be avoided. Therefore, RA is to be withdrawn from the culture medium or its concentration is to be decreased after 3 to 10 days, preferably after 3 to 7 days, from the beginning of the differentiation method. The cells are then cultured in the presence of the TGF-beta inhibitor (preferably SB431542) and the Wnt activator (CHIR99021) without RA or in the decreased concentration of RA for about 1 to about 20 additional days or longer. In some embodiments, the cells start to suffer if exposed to the TGF-beta inhibitor and the Wnt activator for longer than about three weeks.

In some embodiments, RA is withdrawn from the culture medium containing at least one TGF-beta inhibitor, such as SB431542, and at least one Wnt activators, such as CHIR99021, all at once by simply replacing a culture medium containing RA to a culture medium not containing RA. In some alternative embodiments, RA is withdrawn from the culture medium gradually, for example in two to five steps, wherein in each step a culture medium containing a specified amount of RA is replaced with a culture medium containing a lower amount of RA. The duration of each step may vary, for example, from one to three days. In other words, the cells may be cultured in each of the culture media containing a decreased concentration of RA for one to three days before replacing the culture medium to a next culture medium with an even lower concentration of RA or no RA. It also is to be understood, that RA may be withdrawn, all at once or gradually, completely or to a significantly lowered concentration, such as to a concentration decreased by a factor of 10 from RA's initial concentration (i.e., the concentration of RA is reduced to one-tenth of its initial concentration). In an exemplary embodiment, step a) of the present method comprises culturing PCSs in a culture medium comprising 10 μM RA (in addition to at least one TGF-beta inhibitor, such as SB431542, and at least one Wnt activator, such as CHIR99021) for 3 days, followed by step b) wherein the concentration of RA is lowered to 5 μM for the next 1-3 days, further followed by complete omission of RA. In another exemplary embodiment, step a) of the present method comprises culturing PCSs in a culture medium comprising 10 μM RA (in addition to at least one TGF-beta inhibitor, such as SB431542, and at least one Wnt activator, such as CHIR99021) for 3 days, followed by step b) wherein the concentration of RA is first lowered to 5 μM for the next 1-3 days and then to 1 μM for the remaining duration of the differentiation method.

Notably, the present method is preferably carried out in the absence of some agents previously suggested for differentiation of CEC-like cells, such as insulin, EGF, bFGF, Noggin, PDGF, DKK-2, ROCK (Rho-associated kinase) inhibitors such as H-1125 and Wnt inhibitors such as IWP2.

TABLE 1
Outline of an embodiment of the present
differentiation induction method

| | | Duration | Day |
|---|---|---|---|
| Step | Induction agent | in days | count |
| a) | TGF-beta inhibitor (pref. SB431542) + Wnt | 3-10 | 1 |
| | activator (pref. CHIR99021) + retinoic acid | (pref. 3-5) | |
| b) | TGF-beta inhibitor (pref. SB431542) + Wnt | 1-10 | 4-11 |
| | activator (pref. CHIR99021) | | (pref. |
| | | | 4-6) |

In some embodiments, the present differentiation induction method is carried out as an adherent culture, i.e. on a substrate, such as a cell culture bottle or plate, coated with one or more extracellular matrix (ECM) proteins, including both natural extracted and recombinant ECM proteins. Suitable ECM proteins include, but are not limited to, laminins, collagens (e.g. collagen IV), vitronectin, fibronectin, nidogens, proteoglycans, and E-cadherin, as well as isoforms, fragments, and peptide sequences thereof. Non-limiting examples of said ECM isoforms include laminin isoforms such as laminin-511, -521, -322 and -411, while non-limiting examples of said fragments include E8 fragments of said human laminin isoforms. In some preferred embodiments, the cell culture substrate is coated with laminin-521, for example at a concentration range from about 0.5 μg/cm 2 to about 1.5 μg/cm 2 or higher. In some other embodiments, the cell culture substrate is coated with a mixture of collagen IV and laminin-521, for example at concentrations of 5 μg/cm 2 and 0.75 μg/cm 2, respectively.

For clinical acceptance, the CEC-like cells must possess functional features critical for clear vision, corresponding to those of primary CECs. Thus, the CEC-like cells must have a sufficient pumping activity to keep the eyesight clear by pumping fluids out of the corneal stroma. In addition, the CEC-like cells must have sufficient barrier properties as a result of non-leaky tight junctions. As shown in Examples, the CEC-like cells produced by the present differentiation induction method fulfill both of these requirements.

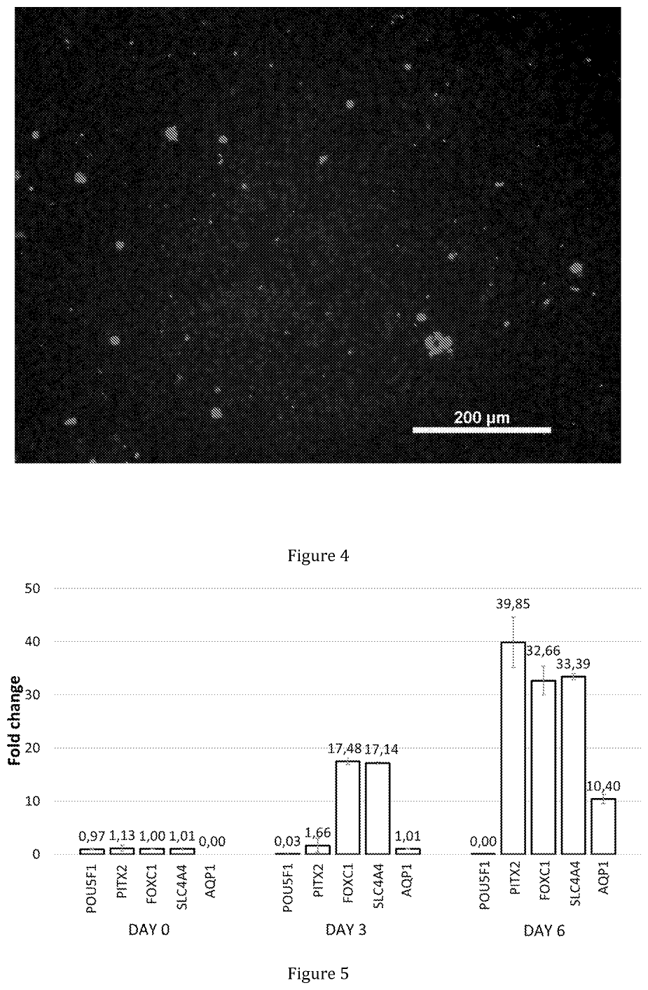

Moreover, for safety reasons, and more specifically to avoid a risk of formation of teratomas or other tumors, it is important that no stem cells are remaining in the differentiated cell population to be used clinically. Indeed, this is the case with the CEC-like cells produced by the present method as demonstrated by lack of OCT3/4 (POU5F1) in both immunofluorescence stainings (FIG. 4) and qPCR (FIG. 5).

### TGF-Beta (TGF-β) Inhibitor

As used herein, with “TGF-beta inhibitor” is referred functionally to a substance capable of inhibiting transforming growth factor β1. Transforming growth factor β1 (TGF-β1) is a member of a large superfamily of pleiotropic cytokines that are involved in many biological activities, including growth, differentiation, migration, cell survival, and adhesion in diseased and normal states. Nearly 30 members have been identified in this superfamily. These are considered to fall into two major branches: TGFβ/Activin/Nodal and BMP/GDF (Bone Morphogenetic Protein/Growth and Differentiation Factor). They have very diverse and often complementary functions. Some are expressed only for short periods during embryonic development and/or only in restricted cell types (e.g. anti-Mullerian hormone, AMH, Inhibin) while others are widespread during embryogenesis and in adult tissues (e.g. TGFβ1 and BMP4). TGF-β1 is a potent regulator in the synthesis of the extracellular matrix (fibrotic factor) and plays a role in wound healing.

In chemical and structural terms, suitable TGF-beta inhibitory function may be found among proteins and small organic molecules. A person skilled in the art is aware of means for isolating proteins from biological matrixes or producing them i.e. by recombinant techniques. Compounds exhibiting TGF-beta inhibitory activity may be found by screening. Preferably, a TGF-beta inhibitor is an organic molecule having a relatively low molar mass, e.g. a small molecule having molar mass less than 800 g/mol, preferably less than 500 g/mol. As a general structure, Formula I, a suitable low molar mass TGF-beta inhibitor may be described as:

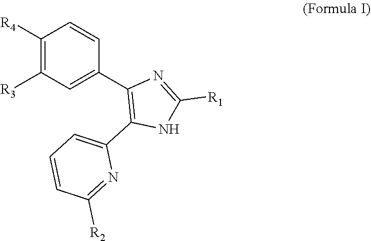

wherein R₁ represents a C₁-C₅ aliphatic alkyl group, carboxylic acid, amide, and R₂ represents a C₁-C₅ aliphatic alkyl, R₃ and R₄ represent aliphatic alkyls including heteroatoms, O or N, which may be linked together to form a 5- or 6-membered hetero ring.

A typical structure comprises a hetero ring having 2 oxygen atoms, when it can be referred to as a small molecule of general formula II:

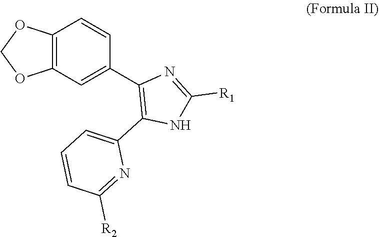

wherein R₁ represents a C₁-C₅ aliphatic alkyl group, an aromatic carboxylic acid or amide, and R₂ represents a C₁-C₅ aliphatic alkyl.

One non-limiting example of such a TGF-beta inhibitor is 4-[4-(1,3-Benzodioxol-5-yl)-5-(2-pyridinyl)-1H-imidazol-2-yl]-benzamidealso known as SB431542, which is commercially available from multiple suppliers and marketed as a selective inhibitor of transforming growth factor-β type I receptor (ALK5), ALK4 and ALK7. Another non-limiting example of specific TGF-inhibitors is 2-(5-benzo[1,3]dioxol-5-yl-2-tert-butyl-3H-imidazol-4-yl)-6-methylpyridine hydrochloride hydrate, also known as SB505124.

However, other small molecules exhibiting TGF-beta inhibitory activity or commercially marketed as TGF-inhibitors may be equally suitable in the context of the present invention. When selecting said TGF-beta inhibitor from substances obtainable by chemical synthesis or recombinant production, a defined medium can be provided. It also complies with requirements of xeno-free and serum-free conditions.

Those skilled in the art can easily determine, using various methods readily available in the art, whether or not a given agent has TGF-beta inhibiting activity or not, and whether it is suitable for use in the present method.

In some preferred embodiments, the concentration of the TGF-beta inhibitor, such as SB431542, in the induction medium is from about 1 μM to about 100 μM, preferably from about 1 to about 30 μM, more preferably from about 5 μM to about 15 μM, and still more preferably about 10 μM.

### Wnt Activator

The Wnt (Wingless-related integration site) family of protooncogenes consists of at least 16 known members which encode secreted signaling proteins that are involved in oncogenesis and several other developmental processes, such as regulation of cell fate and embryogenesis. As used herein, the term “Wnt activator” refers to a substance capable of activating Wnt signaling pathway. Both protein and small molecular Wnt activators are known in the art.

Glycogen synthase kinase 3 (GSK3) inhibitors are an exemplary class of preferred Wnt activators for use in the present invention. CHIR99021, which is commercially available from multiple providers, is the most selective inhibitor of GSK3 and, thus, a particularly preferred Wnt activator for use in the present invention. Further GSK3 inhibitors include, but are not limited to, SB-216763, BIO(6-bromoindirubin-3′-oxime), LY2090314 and lithium chloride, all of which are commercially available. In some preferred embodiments, the amount of the GSK3 inhibitor, such as CHIR99021, in the differentiation medium is from about 1 μM to about 15 μM, preferably from about 1 μM to about 10 μM, more preferably from about 1 μM to about 5 μM, and still more preferably about 4 μM.

Another class of Wnt activators envisaged to be suitable for use in the present invention is the R-spondin protein family, the four members of which, designated as R-spondin-1, R-spondin-2, R-spondin-3 and R-spondin-4, are secreted agonists of the canonical Wnt/β-catenin signaling pathway.

In some embodiments, the Wnt activator is R-spondin-1. Preferred concentration ranges include from about 100 ng/ml to about 2 μg/ml, from about 500 ng/ml to about 2 μg/ml, and preferably about 1 μg/ml.

RS-246204 is a small molecule R-spondin-1 substitute, which is also envisaged to be suitable for use in the present invention. Preferred concentration ranges include from about 6.25 μM to about 200 μM, and from about 25 μM to about 50 μM.

Those skilled in the art can easily determine using various methods readily available in the art, whether or not a given agent has Wnt-activating properties or not, and whether it is suitable for use in the present method. A compound can be tested for its ability to act as Wnt activators e.g. by a commercial test kit, LEADING LIGHT® Wnt Reporter Assay Starter Kit available from Enzo.

### Retinoic Acid

As used herein, the term “retinoic acid” (RA) encompasses all isomers of retinoic acid, including the major all-trans-retinoic acid (ATRA; generally referred to as retinoic acid for the sake of simplicity of expression), and its minor isomers such as 9-cis-retinoic acid, 11-cis-retinoic acid and 13-cis-retinoic. In some embodiments, retinoic acid to be employed is ATRA.

ATRA is a metabolite of vitamin A1 (all-trans-retinol) derived through two consecutive enzymatic reactions catalyzed by different sets of dehydrogenases. ATRA plays important roles in cell growth, differentiation, and organogenesis.

In some embodiments, RA, preferably ATRA, is used in step a) of the present method in a concentration ranging from about 1 μM to about 20 μM, preferably about 10 μM, whereas RA, preferably ATRA, is used in step b) of the present method in a concentration ranging from 0 to about 1 μM. As explained above, the concentration of RA used in step a) may be lowered to the concentration of RA used in step b) either gradually or all at once. Retinoic acid is commercially available from different sources.

### Cell Culture Medium

In the present method and its various embodiments, basically any cell culture medium suitable for differentiating stem cells may be used as a basal medium to be supplemented with the present differentiation induction agents. As used herein, the term “basal medium” refers to a cell culture medium composed of components, including amino acids, glucose and ions such as calcium, magnesium, potassium, sodium and phosphate, as is well known in the art. Non-limiting examples of commercially available basal media suitable for use in the present method include KnockOut Dulbecco's Modified Eagle's Medium (KO-DMEM), Dulbecco's Modified Eagle's Medium (DMEM), Minimal Essential Medium (MEM), Basal Medium Eagle (BME), RPMI 1640, F-10, F-12, Glasgow's Minimal Essential Medium (G-MEM), Iscove's Modified Dulbecco's Medium and any combinations thereof.

In addition to the differentiation induction agents, the basal medium may be supplemented with ingredients used in practically every cell culture medium including antibiotics, L-glutamine, and serum, serum albumin or a serum replacement, preferably a defined serum replacement. Further supplements common in the art may also be applied, unless they are known to direct differentiation of PSCs towards tissues other than conical endothelium. In some embodiments, the induction medium to be used in the present method does not contain ingredients other than the induction agents, basal medium, antibiotics, L-glutamine, and a defined serum replacement.

When in use or when ready for use, the cell culture medium comprises appropriate essential supplements set forth above. However, according to a common practice in the field, the ingredients for a medium may be provided as a concentrate comprising said components or a set of vials from which an appropriate combination is prepared prior to use in a laboratory according to instructions provided. Often, culture medium is diluted and prepared to the final composition immediately before use. Therefore, it is understood that any stock solution or preparation kit suitable for use in such immediate preparation may be used for obtaining cell culture media to be used in the present method.

For better clinical acceptance, all culture media to be used in the present method and its various embodiments are preferably substantially xeno-free, substantially serum-free, or substantially defined, more preferably combinations of these, and most preferably substantially xeno-free, substantially serum-free, and substantially defined at the same time. With “substantially” is meant herein that unintentional traces are irrelevant, and what is under clinical or laboratory regulations considered and accepted as xeno-free, serum-free or defined, applies here as well.

As used herein the term “xeno-free” refers to absence of any foreign material or components. Thus, in case of human cell culture, this refers to conditions free from non-human animal components. In other words, when xeno-free conditions are desired for production of corneal cells for human use, all components of any cell culture media must be of human or recombinant origin.

Traditionally, serum, especially fetal bovine serum (FBS), has been valued in cell cultures providing essential growth and survival components for in vitro cell culture of eukaryotic cells. It is produced from blood collected at commercial slaughterhouses from cattle bred to supply meat destined for human consumption. “Serum free” indicates that the culture medium contains no serum, either animal or human.

Defined medium is valuated when there are contradictions for use of undefined media, e.g. “conditioned medium”, which refers to spent media harvested from cultured cells containing metabolites, growth factors, and extracellular matrix proteins secreted into the medium by the cultured cells. Undefined media may be subject to considerable dissimilarities due to natural variation in biology. Undefined components in a cell culture compromise the repeatability of cell model experiments e.g. in drug discovery and toxicology studies. Hence, “defined medium” or “defined culture medium” refers to a composition, wherein the medium has known quantities of all ingredients.

Typically, serum that would normally be added to culture medium for cell culture is replaced by known quantities of serum components, such as, e.g., albumin, insulin, transferrin and possibly specific growth factors (e.g., basic fibroblast growth factor, transforming growth factor or platelet-derived growth factor).

A chemically defined medium is a growth medium in which all of the chemical components are known. A chemically defined medium is entirely free of animal-derived components and represents the purest and most consistent cell culture environment. By definition, chemically defined media cannot contain fetal bovine serum, bovine serum albumin or human serum albumin as these products are derived from bovine or human sources and contain complex mixes of albumins and lipids.

Chemically defined media differ from serum-free media in that bovine serum albumin (BSA) or human serum albumin (HSA) is replaced with either a chemically defined recombinant version (which lacks the albumin associated lipids) or a synthetic chemical, such as the polymer polyvinyl alcohol, which can reproduce some of the functions of BSA/HSA.

In some embodiments, the cell culture medium comprises a serum replacement formulation. One example is described in Raj ala et al. 2010, which is incorporated here as reference, describing a xeno-free serum replacement applicable in the context of the present invention. Further non-limiting examples of suitable serum replacements include KnockOut™ Serum Replacement (Ko-SR) and its xeno-free version KnockOut™ SR XenoFree CTS™, both commercially available from Life Technologies.

### Therapeutic Use and Pharmaceutical Compositions

The present invention also provides a method of treating corneal endothelial dysfunction in a subject in need thereof. The method comprises transplanting an efficient amount of CEC-like cells produced in accordance with the present invention to said subject intraocularly.

In accordance with the above, the present invention also provides CEC-like cells produced in accordance with the present invention for use in treating corneal endothelial dysfunction.

As used herein, the term “subject” refers to any mammals, preferably humans.

As used herein, the term “treatment” or “treating” involves the administration of the present CEC-like cells to a subject by intraocular transplantation for purposes which may include ameliorating, lessening, inhibiting or curing of the corneal endothelial dysfunction.

As used herein, the term “corneal endothelial dysfunction” refers to any disorder or condition affecting corneal endothelial cells. Non-limiting examples of such disorders or conditions include Fuchs' endothelial corneal dystrophy, bullous keratopathy, congenital hereditary endothelial dystrophy, posterior polymorphous dystrophy, iridocorneal endothelial syndrome, corneal edema, corneal leukoma, corneal endothelial inflammation, chemical burns and surgical or other trauma. The main treatment for these disorder and condition is replacement of the abnormal corneal layers with normal donor tissue.

As used herein, the term “efficient amount” refers to an amount of CEC-like cells by which harmful effects of the corneal endothelial dysfunction are, at a minimum, ameliorated.

The CEC-like cells for use in therapy may be either allogenic or autologous.

Amounts and regimens for transplantation of the present CEC-like cells can be determined readily by those with ordinary skill in the clinical art of treating eye diseases, especially corneal endothelial dysfunction. Generally, dosing will vary depending on considerations such as: age, gender and general health of the subject to be treated; kind of concurrent treatment, if any; severity and type of disease or condition in question; causative agent of the disease and other variables to be adjusted by the individual physician.

For transplantation, the CEC-like cells are provided in a pharmaceutical preparation. As used herein, the term “pharmaceutical preparation” refers broadly to a preparation of CEC-like cells and one or more physiologically acceptable components such as solutions, carriers, adjuvants and/or excipients. Preferably, the components are sterile.

As used herein, the terms “physiologically acceptable” and “pharmaceutically acceptable” are interchangeable and refer to a material that is suitable for administration to a subject, preferably a human subject, without undue adverse side effects such as toxicity, significant irritation and/or allergic responses. In other words, the benefit/risk ratio must be reasonable. Moreover, the physiologically acceptable components must not harm the CEC-like cells.

In some embodiments, the CEC-like cells are provided in a physiologically acceptable solution. Suitable solutions include, but are not limited to, phosphate buffered saline and other isotonic aqueous buffer solutions such as Ringer's solution.

For transplantation by direct intraocular injection, preferably into the anterior chamber, the CEC-like cells are provided as a suspension in a physiologically acceptable solution. Density of the cells in the suspension may vary as desired but is typically from about 1×10⁶ to about 5×10⁶ cells/ml. In some embodiments, the suspension may comprise carriers, such as microparticles, microbeads, gel-like matrices and the like.

In some embodiments, the CEC-like cells are provided as a monolayer of CEC-like cells. The monolayer may have a cell density ranging from about 1000 cells/mm² to about 6000 cells/mm², preferably from about 2000 cells/mm² to about 5000 cells/mm², and preferably from about 3000 cells/mm² to about 4000 cells/mm². In some embodiments, said monolayer is provided on a physiologically acceptable carrier membrane. Usually, a pharmaceutical preparation comprising a monolayer of CEC-like cells on a carrier membrane also comprises a physiologically acceptable solution so as to maintain the viability of the cells and to prevent them from drying.

The carrier membrane is not particularly limited provided it can support the CEC-like cells, is transparent and otherwise suitable for intraocular transplantation. Non-limiting examples of suitable carrier membranes include fibrin-based matrixes, endothelium-decellularized corneal buttons, decellularized Descemet's membrane, human or non-human animal-derived fresh corneal stromal discs, decellularized amniotic membrane, fish scale-derived scaffolds, membranes prepared from collagen, gelatin, silk fibroin, cellulose and the like, synthetic polymer materials such as polystyrene, polyester, polycarbonate, poly(N-isopropylacrylamide) and the like, semi-synthetic hydrogel gelatin-methacryloyl, biodegradable polymer materials such as polylactic acid, polyglycolic acid, poly(lactic-co-glycolic acid), polycaprolactone and the like, and mineral based hydroxyapatite.

In some embodiments, the carrier membrane has lateral dimensions corresponding to the desired lateral dimensions of the final carrier membrane to be used for intraocular transplantation. In other words, the size and shape of the carrier membrane may correspond to those of a carrier membrane that is intended to be used as such without cutting or otherwise resizing the membrane before use. In some other embodiments, the lateral dimensions of the carrier membrane may be larger than those of the final carrier membrane to be used. In such embodiments, size and shape of the membrane is to adjusted to the desired size and shape before use by any suitable technique available in the art including but not limited to cutting and punching e.g. by a biopsy punch or a trephine. Typically, the carrier membrane has a circular shape having a diameter from about 5 mm to about 10 mm, preferably from about 7.5 mm to about 8 mm. In some embodiments, the final size and shape of the carrier membrane correspond to those of human cornea. Usually, the thickness of the carrier membrane is less than about 100 μm, preferably ranging from about 5 μm to about 50 μm.

### Use in Drug Development and Basic Research

The present invention may also be applied in drug development and basic research. For example, CEC-like cells produced in accordance with the present invention may be employed for gaining mechanistic insight into a candidate compound's action by studying its biological and pharmacological effects, including for example biomolecular interactions and pathways impacted by the compound. In addition to mechanistic studies, the cells may be employed, for example, in drug screening or toxicological studies.

In some embodiments, the aim of the drug development may be to identify candidate compounds for the presence or absence of a pharmacological effect on CEC-like cells. The presence or absence of a pharmacological effect may be determined based on various readouts including, but not limited to, a change in marker expression, morphology, pumping activity, viability, proliferation rate, and secretion of proteins, cytokines or extracellular matrix components, as compared to corresponding effects in control cells, such as CEC-like cells contacted with a control compound or CEC-like cells not contacted with any test compound.

As readily understood by those skilled in the art, the readout employed depends on the pharmacological effect whose presence or absence is to be determined. The pharmacological effect to be determined may be a desired pharmacological effect or an adverse effect. Thus, CEC-like cells may be used not only in screening for desired pharmacological effects but also in screening for adverse pharmacological effects or any side effects such as toxicity.

Accordingly, the invention provides a preparation comprising CEC-like cells of the invention and one or more additional components such as cell-compatible solutions, carriers, adjuvants and/or excipients, for example for the purposes mentioned above. In some embodiments, the carrier is a carrier membrane set forth above and the CEC-like cells are provided on the carrier membrane. As used herein, the term “cell-compatible” refers broadly to a component that is suitable for use in maintaining living cells without significant adverse effects to the cells.

### EXPERIMENTAL PART

While the present invention will be described in further detail below referring to examples, it is not intended that the present invention be limited to the examples.

### Materials and Methods

### Cells

Embryonic stem cell (hESC) line Regea08/017 was derived as described and characterized by Skottman, In Vitro Cell Dev Biol Anim 2010; 46(3-4):206-9.

Human induced pluripotent stem cells (hiPSC) were generated from peripheral blood mononuclear cells using CytoTune™-iPS 2.0 Sendai Reprogramming Kit according to the manufacturer's instructions.

Human primary CECs were obtained from donated corneas not suitable for clinical use.

All cell experiments were carried out under appropriate statements from the ethics committee of Pirkanmaa hospital district concerning the use hESC lines from surplus human embryos for research (R05116) and production of hiPSC lines for ophthalmic research including patients and healthy voluntary donors (R16116). Furthermore, together with Regea tissue bank, Tampere University has an ethical approval for research use of human donor corneas unsuitable for transplantation (R11134).

Free and fully informed consent was received for each donation after written and oral description of the use of the donated tissue.

Differentiation of Human Pluripotent Stem Cells (hPSC) into Corneal Endothelial Cells (CEC)

Embryonic (hESC) and induced pluripotent stem cells (iPSC) were used to generate CECs. Briefly, hPSCs were seeded on Laminin 521 (Biolamina) coated CellBind 6/12 well-plates (Corning) in 10 000-60 000 cell/cm 2 cell density. The cells were cultured in Essential 8 Flex medium (Thermo Fisher) for 24h. On day 1, Essential 8 flex medium was replaced with an induction medium supplemented with 10 μM SB431542 (Stemcell), 4 μM CHIR99021 (Stemcell) and 10 μM retinoic acid (RA, Sigma-Aldrich). Base of the induction medium consisted of KO-DMEM, 15% Knock-out serum replacement, 2 mM GlutaMax-I, 0.1 mM 2-mercaptoethanol (all from Thermo Fisher), 1% Non-essential Amino Acids, 50 U/ml Penicillin/Streptomycin. For days 4-7, RA was removed completely or the concentration was gradually lowered, for example by using 10 μM RA for 3 days, followed by lowering of the concentration to 5 μM for the next 1-3 days, further followed by complete omission of RA or lowering the concentration of RA to 1 μM for the remaining duration of the differentiation method. After day 9, CEC-like cells were formed, and harvested for analyses.

Characterization of hPSC-Derived CECs by Immunocytochemistry

The hPSC-derived CECs were analyzed by immunocytochemistry. Briefly, the cells were fixed with 1 or 4% paraformaldehyde (PFA, Sigma-Aldrich) for 15 minutes. Next, the cells were permeabilized for 10 minutes with 0.1% Triton X-100 (Sigma-Aldrich) followed by blocking with 3% bovine serum albumin (BSA) for 1 hour. Then the cells were first incubated with 1:400 zona occludens-1 (ZO-1; Thermo Fisher), 1:200 alpha 1 sodium potassium ATPase (Na+/K+-ATPase, Abcam) and 1:400 CD166 (BD Biosciences) primary antibodies overnight at 4° C. The cells were next treated with 1:800 Donkey anti-Rabbit IgG Secondary Antibody, Alexa Fluor 488 (Thermo Fisher) against ZO-1; 1:800 Donkey anti-Mouse IgG Secondary Antibody, Alexa Fluor 568 (Thermo Fisher) against Na+/K+-ATPase and CD166; and 1:800 Donkey anti-Goat IgG Secondary Antibody, Alexa Fluor 568 (Thermo Fisher) against OCT-3/4. The nuclei were counterstained with 4′,6-diamidine-2′-phenylindole dihydrochloride (DAPI, Vector laboratories). The images of mounted cells were captured using a fluorescence microscope (Olympus IX51; Olympus, Tokyo, Japan) and prepared using image editing software (Adobe Photoshop CC 2018; Adobe Systems)

### Phase Contrast Microscopy

Phase contrast light microscope Nikon Eclipse TE2000-S with a DS-Fi1 camera (Nikon Corp. Tokyo, Japan) was used to capture images of the cell morphology.

qPCR

Total RNA was extracted from undifferentiated hPSCs (d0), and from 3 time points during the time of hCEC induction (d3, d6 and d9) with Rneasy Minikit Plus (Qiagen). RNA concentration of each sample was determined using NanoDrop-1000 spectrophotometer (NanoDrop Technologies). From each RNA sample 400 ng were used to synthesize cDNA using the High-Capacity cDNA RT kit (Applied Biosystems). The resulting cDNA samples were analyzed with qPCR using sequence-specific TaqMan Gene Expression Assays (Thermo Fisher) for OCT4 (Hs00999632_g1), PITX2 (Hs01553179_m1), AQP1 (Hs01028916_m1), SLC4A4 (Hs00186798_m1), and FOXC1 (Hs00559473_s1). All samples were run as triplicate reactions with the 7300 Real-Time PCR system (Applied Biosystems). Results were analyzed with the 7300 System SDS Software (Applied Biosystems) and Microsoft Excel. Based on the cycle threshold (CT) values given by the software, the relative quantification of each gene was calculated by applying the −2ΔΔCt method (Livak and Schmittgen, 2001). Results were normalized to GAPDH (Hs99999905_m1), with the undifferentiated hPSCs as the calibrator to determine the relative quantities of gene expression in each sample.

### Results

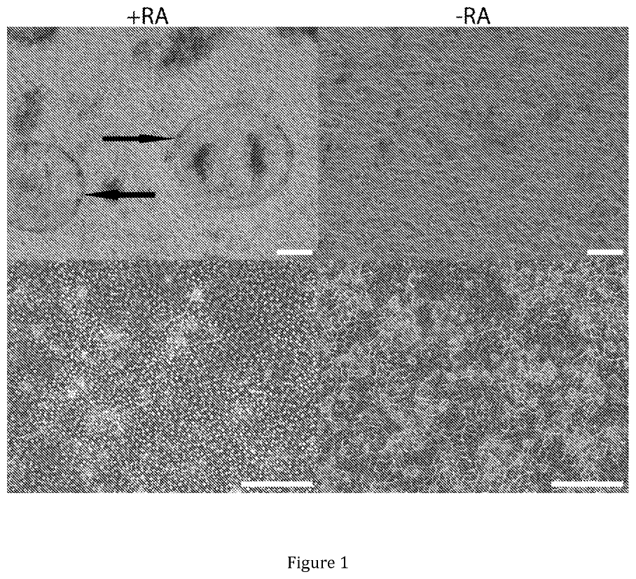

In earlier studies, SB431542 and CHIR99021 have been used in the differentiation of neural crest cells, followed by addition of various induction cocktails such as B27, PDGF-BB and DKK2 (Wagoner et al. 2018). The present inventors found that corneal endothelial cell (CEC)-like cells can be differentiated in a simpler and faster manner by adding retinoic acid (RA) in the beginning of the differentiation for 3-5 days with SB431542 and CHIR99021. After 7 days of differentiation, the morphology of the cells become polygonal and close to native hexagonal appearance. The presence of RA for the first 3-5 days caused the cell monolayer to form dome-like structures which indicates barrier properties and pumping activity in the cells. Cell cultures without RA did not have these features (FIG. 1).

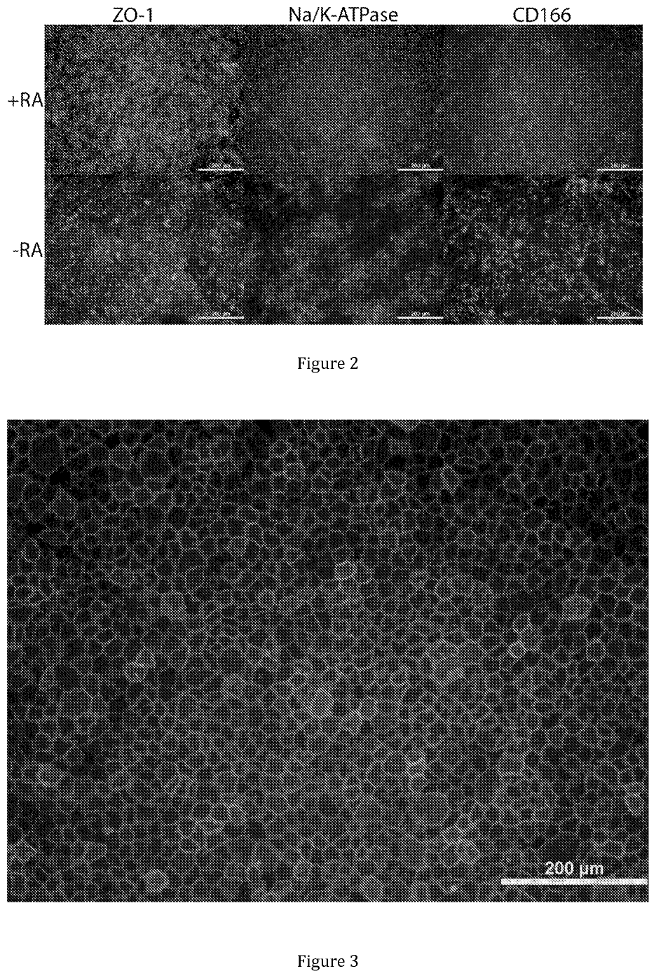

To further characterize the CEC-like cells, immunofluorescence stainings were performed to verify correct protein expression and localization. The cells were immunostained for fundamental CEC markers, Zona Occludens 1 (ZO-1), a tight junction marker, Na+/K+ATPase, an important pump protein for CEC, and CD166, a surface marker that has been noted to be relatively specific for CECs (FIG. 2). ZO-1 localized in the boundaries of the cells, demonstrating that the cells were tightly adherent (FIG. 2). Cells with RA resembled closely to cultured human primary CECs when comparing the Na+/K+ATPase immunostainings (FIG. 3). CD166 was localized on the surface in the same areas as Na+/K+ATPase (FIG. 2).

Lack of OCT3/4 (POU5F1) expression was observed in both immunofluorescence stainings (FIG. 4) and qPCR (FIG. 5), indicating the absence of stem cells in the differentiated cell population.

1. A method of producing corneal endothelial cell (CEC)-like cells from pluripotent stem cells, comprising
a) culturing the pluripotent stem cells in the presence of at least one transforming growth factor beta (TGF-beta) inhibitor, at least one Wingless-related integration site (Wnt) activator and retinoic acid, and
b) culturing cells from step a) in the presence of at least one TGF-beta inhibitor and at least one Wnt activator, but in the absence of or gradually decreasing concentration of retinoic acid,
thereby producing CEC-like cells.

2. The method according to claim 1, wherein the TGF-beta inhibitor is selected from the group consisting of SB431542 and SB505124.

3. The method according to claim 1, wherein the Wnt activator is selected from the group consisting of glycogen synthase kinase 3 (GSK3) inhibitors and proteins of R-spondin family.

4. The method according to claim 3, wherein the GSK3 inhibitor is CHIR99021.

5. The method according to claim 1, wherein the retinoic acid is all-trans-retinoic acid (ATRA).

6. The method according to claim 1, wherein the duration of step a) is from 3 to 10 days.

7. The method according to claim 1, wherein the duration of step b) is from 1 to 20 days.

8. The method according to claim 2, wherein the concentration of SB431542 or SB505124 is from about 1 μM to about 100 μM.

9. The method according to claim 4, wherein the concentration of CHIR99021 is from about 1 μM to about 15 μM.

10. The method according to claim 1, wherein the concentration of retinoic acid in step a) is about 10 μM.

11. The method according to claim 1, wherein the concentration of retinoic acid is gradually decreased to the concentration of 0-1 μM in step b).

12. The method according to claim 1, wherein the pluripotent stem cells are selected from the group consisting of induced pluripotent stem cells and embryonic stem cells, with the proviso that if human embryonic stem cells are used, the method does not involve destruction of human embryos.

13. The method according to claim 1, wherein the cells are cultured on a cell culture substrate coated with one or more ECM proteins selected from the group consisting of laminins, collagens, vitronectin, fibronectin, nidogens, proteoglycans, and E-cadherin, and isoforms, fragments, and peptide sequences thereof.

14. The method according to claim 13, wherein the ECM protein is laminin-521.

15. The method according to claim 1, wherein the CEC-like cells express one or more markers selected from the group consisting of zonula occludens-1 (ZO-1), ATPase Na+/K+ transporting subunit alpha 1 (ATP1A1), solute carrier family 4 member 4 (SLC4A4), CD166, aquaporin 1 (AQP1), paired like homeodomain 2 (PITX2), and forkhead box C1 (FOXC1).

16. CEC-like cells obtainable by the method of claim 1.

17. The CEC-like cells according to claim 16, wherein the cells are human CEC-like cells.

18. Use of TGF-beta inhibitor, Wnt activator and retinoic acid in combination for inducing CEC-like cells from pluripotent stem cells.

19. CEC-like cells according to claim 16 for use in the treatment of corneal endothelial dysfunction or for use in drug development.

20. A preparation comprising CEC-like cells according to claim 16 and a solution, carrier, adjuvant and/or excipient.

21. The preparation according to claim 20, wherein the CEC-like cells provided as a cell monolayer.
